(19) United States
(12) Patent Application Publication
(10) Pub. No.: US 2006/0004320 A1
(43) Pub. Date: Jan. 5, 2006

(54) Applicator with low placement indicia on the plunger

(75) Inventors: Charles John Berg JR., Wyoming, OH (US); Diana Lynne Gann, Lebanon, OH (US); Thomas Ward Osborn III, Clifton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/881,888

(22) Filed: Jun. 30, 2004

(51) Int. Cl. A61F 13/20

(52) U.S. Cl. 604/15

(57) ABSTRACT

An applicator device capable of housing a tampon is shown that includes an insertion member and a plunger. The insertion member includes an insertion end opposed to a gripper end. The plunger is slideably configured within the insertion member. The plunger includes an outside diameter, longitudinal axis, a first end opposed to a second end and an indicia of low placement. The indicia of low placement is located on between the first end and the second end of the plunger.

### FIELD OF THE INVENTION

This invention relates to an applicator, particularly, to a tampon applicator that comprises an indicia of low placement on the plunger of the applicator.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators in their simplest form comprise a pair of coaxial hollow cylinders or tubes. The larger outer tube serves as a vaginal insertion tube that includes the tampon, while the smaller inner tube, or plunger, ejects the tampon from the outer tube.

It has been recognized that “push” type tampon applicators have certain deficiencies. It is also recognized that the position of the tampon within the vaginal cavity can directly impact the tampon's efficiency of reducing and/or eliminating leakage. For example, conventional “push” type applicators can place a tampon too high in the vaginal cavity above the major path of fluid flow, so that menstrual fluids can pass by the tampon without being absorbed. Alternatively, tampon applicators can place a tampon too low in the vaginal cavity, which can lead to discomfort due to the pressure exerted from the sphincter muscles of the wearer against the tampon. Another highly undesired risk of placing the tampon too low is accidental expulsion of the tampon.

To solve the above problem of positioning the tampon, it would be beneficial to provide a tampon applicator which properly places the tampon at a desired position in the vaginal cavity.

### SUMMARY OF THE INVENTION

The present invention comprises an applicator device capable of housing a tampon. The applicator comprises an insertion member and a plunger. The insertion member comprises insertion end opposed to a gripper end. The plunger is slideably configured with the insertion member. The plunger comprises a longitudinal axis, a first end opposed to a second end and an indicia of low placement. The indicia of low placement is located on between the first end and the second end of the plunger.

### BRIEF DESCRIPTION OF THE DRAWINGS

FIG. 1 is a side view of one embodiment of the applicator of the present invention with a cross section of the insertion member, also comprising a tampon.

FIG. 2 is a side view of another embodiment of the applicator of the present invention with a cross section of the insertion member, also comprising a tampon having a withdrawal member.

FIG. 3 is a side view of another embodiment of the applicator of the present invention with a cross section of the insertion member, also comprising a tampon.

FIG. 4 is a side view of another embodiment of the applicator of the present invention with a cross section of the insertion member, also comprising a tampon having a withdrawal member.

FIG. 5 is a side view of another embodiment of the applicator of the present invention in a pre-insertion position with a cross section of the insertion member, also comprising a tampon having a withdrawal member

### DETAILED DESCRIPTION OF THE INVENTION

As used herein “applicator” refers to a device or implement that facilitates the insertion of a tampon, medicament, treatment device, visualization aid, or other into an external orifice of a mammal, such as the vagina, rectum, ear canal, nasal canal, or throat. Nonlimiting specific examples of such include any known hygienically designed applicator that is capable of receiving a tampon can be used for insertion of a tampon, including the so-called telescoping, tube and plunger and the compact applicators, an applicator for providing medicaments to an area for prophylaxis or treatment of disease, a spectroscope containing a microcamera in the tip connected via fiber optics, a speculum of any design, a narrow hollow pipe for guiding surgical instruments, and the like.

The term “expelled” as used herein, is the position of the tampon after the tampon is fully or partially forced out of the tampon applicator. It follows, that as used herein, “expulsion,” is the act of or partially forcing the tampon out of the tampon applicator.

As used herein, the term “hymenal ring” refers to the demarcation between the vaginal cavity and external genitalia and is identified by the position of the hymen or residual tissue of the hymen.

The term “indicia,” as used herein, refers to a mark, demarcation, point, or cue. Recognizing the dictionary definition of indicia is a plural noun, as used herein, the term is intended to include one or a plurality of marks, demarcations, points or cues.

As used herein, the term “low placement” refers to a vertical position of the tampon inside the vaginal cavity, wherein the insertion end of the tampon is positioned generally below the cervix and the withdrawal end of the tampon is positioned generally above the hymenal ring. The withdrawal end of the tampon does not include any overwrap, secondary absorbent member, or withdrawal cord which extends beyond the tampon's main absorbent material.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

As used herein the term “tampon,” refers to any type of absorbent structure that is inserted into the vaginal canal or other body cavities for the absorption of fluid therefrom, to aid in wound healing, or for the delivery of active materials, such as medicaments, or moisture. Generally, there are two types of tampons, self-sustaining and deformable, such as fluid permeable bag tampons. Tampons are generally “self-sustaining” in that they will tend to retain their general shape and size before use. Thus, when a tampon pledget has been compressed and/or shaped such that it assumes a general shape and size, which is vaginally insertable, absent external forces. It will be understood by one of skill in the art that this self-sustaining shape need not, and preferably does not persist during actual use of the tampon. That is, once the tampon is inserted and begins to acquire fluid, the tampon can begin to expand and can lose its self-sustaining form. The tampon can be compressed into a generally cylindrical configuration in the radial direction, axially along the longitudinal axis or in both the radial and axial directions. While the tampon can be compressed into a substantially cylindrical configuration, other shapes are possible. These can include shapes having a cross section that can be described as rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A self-sustaining tampon for use for low placement is less than about60 mm in length and typically from about 30 mm to about 56 mm in length, or 35 mm to about 48 mm in length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 mm wide corresponding to the largest cylindrical cross section. The width can vary along the length of the self-sustaining tampon. The second type of tampon is a “deformable, fluid-permeable bag tampon”. These deformable, fluid-permeable bag tampons are typically not compressed and consists of a fluid permeable bag containing small pieces of absorbent material such as chips, spheres, or fibers. These tampons readily deform with a force of less than about 3 psi.

As used herein, the terms “vaginal cavity,” “within the vagina,” and “vaginal canal” refer to the internal genitalia of the human female in the pudendal region of the body. The term “vaginal canal” is not intended to include the interlabial space including the floor of the vestibule. The externally visible genitalia generally are not included within the term “vaginal canal” as used herein.

FIG. 1-FIG. 5 show the applicator 10 of the present invention. FIG. 1 illustrates is a side view of one embodiment of the applicator 10A of the present invention with a cross section of the insertion member 20A, and a plunger 30A. The insertion member 20A is adapted to house the tampon 36. The insertion member 20A comprises a hollow interior, which in FIG. 1 is filled by the tampon 36, an insertion end 22 opposed to a gripper end 24. The plunger 30A in FIG. 1 comprises a longitudinal axis L, and a first end 26 opposed to a second end 28. The plunger 30A can also comprise one or multiple indicia of low placement 40A that are located between the first end 26 and the second end 28. Typically, the indicia of low placement 40A are proximate to the center of the plunger 30A between the first end 26 and the second end 28. The indicia of low placement 40A will not typically be located at either the first end 26 or second end 28 of the plunger 30. The plunger 30A is slideably configured within the inside diameter of the insertion member 20A. In addition, the first end 26 is the portion of the plunger 30A which contacts the tampon 36 housed within the insertion member 20. The second end 28 is the portion of the plunger 30A in which a force along the longitudinal axis L is applied to expel the tampon 36 through the insertion member 20A.

To use the applicator, the consumer will typically position the insertion end 22 of the insertion member 20A beyond the labia majora and grasp the insertion member 20A at the gripper end 24. Next, the consumer will plunge the applicator 10. As used herein the term “plunge” or “plunging” refers to an action in the step in the process of insertion of the tampon 36 that results in the second end 28 of the plunger 30A, which is slideably configured within the inside diameter of the insertion member 20A, to be in closer proximity towards the gripper end 24 of the insertion member 20A, such that the indicia of low placement 40A on the plunger 30A is flush with the gripper end 24 of the insertion member. During the plunge, the indicia of low placement 40 is a cue for the consumer to stop moving or sliding the plunger 30A towards the gripper end 24 of the insertion member 30A, so that tampon 36 is placed in the desired position within the vagina.

The indicia of low placement 40A can be any demarcation, point, cue in the insertion process which exhibits a sharp or gradual sensorial impression, including visual, auditory, or tactile so as to signify when the desired placement of the tampon 36 will be attained. The indicia 40A are optionally modifiable by the user so as to conform to a predetermined level of insertion ideal for that person. In that vein, there can be more than one indicia of low placement 40A which correspond to various insertion depths. In such a case, the different indicia of low placement 40A will be a different shape or color corresponding to a specific depth of insertion, in order for the user to distinguish and choose the appropriate depth of insertion for their uses.

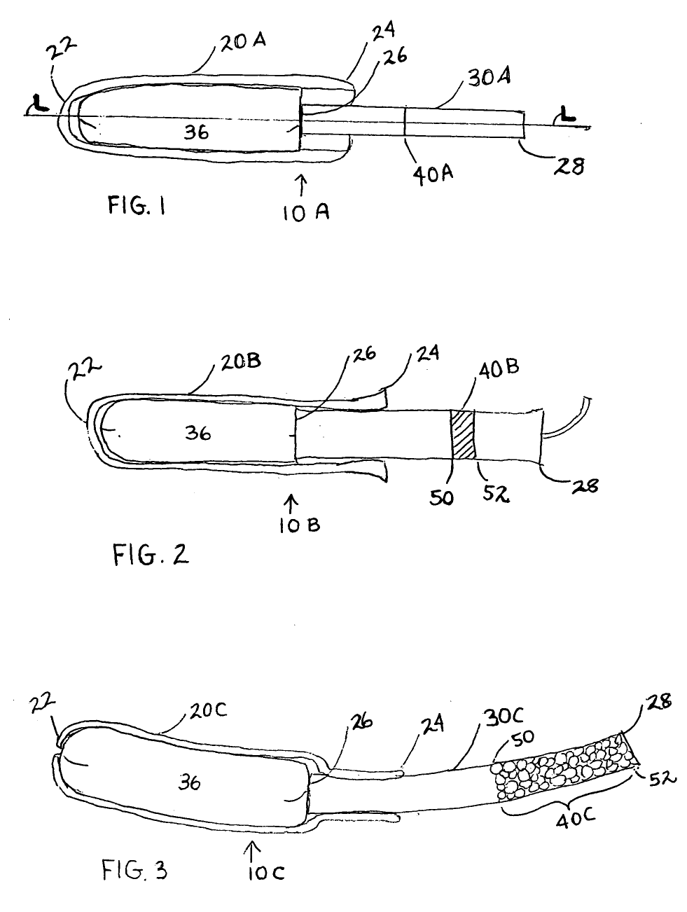

For example, the indicia of low placement 40A can be visual indicia that can comprise a single line, color boundary, a color difference, a pattern difference (including surface pattern difference), or can be a point where the two colors, two patterns, or a pattern and a color meet between the first end 26 and the second end 28 of the plunger 30A. Embodiments of visual indicia for low placement 40A, 40B and 40C are shown in FIG. 1-3. In FIG. 1 the indicia of low placement 40A is in the form of a thin solid line encircling the perimeter of the plunger 30A. In FIG. 2 the indicia for low placement 40B is a wide band of diagonal stripes encircling the plunger 30B located between the first end 26 of the plunger 30B and the second end 28 of the plunger 30B. The indicia for low placement 40B, as depicted in FIG. 2, has a starting point 50 and an ending point 52. As illustrated in FIG. 3, the indicia for low placement 40C is difference in the texture or pattern. The insertion member 20C has a smooth texture or lacks a pattern from the first end 26 of the plunger 30C to the starting point 50 of the indicia for low placement 40C and has a pebbled pattern or texture from the starting point 50 of the indicia for low placement 40B to the ending point 52 of the indicia for low placement 40C proximate to the second end 28 of the plunger 20C. Note that the indicia of low placement 40B and 40C, as illustrated in FIG. 2 and FIG. 3, have a starting point 50 and an ending point 52. Depending on the type and width, the indicia for low placement 40 can comprise a starting point 50 and ending point 52. In some cases, the starting point 50 and ending point 52 can be coexistent or can be so close in proximity as to not be distinguishable from the indicia for low placement 40 itself, such as the thin solid line indicia 40A illustrated in FIG. 1.

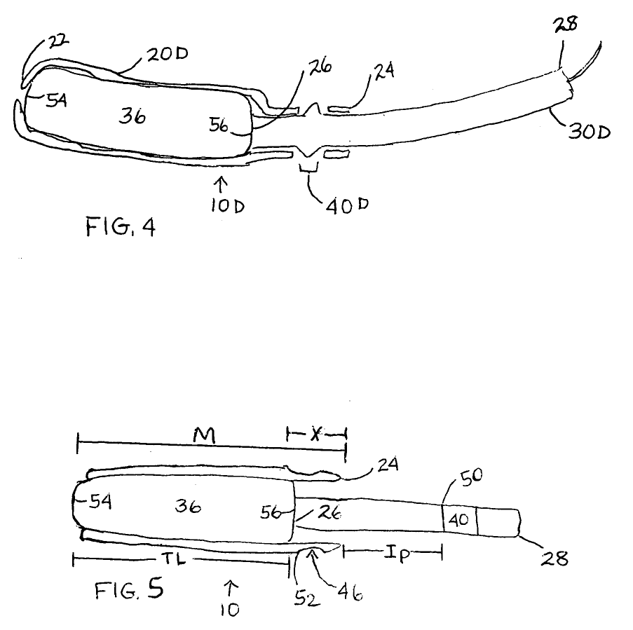

The indicia of low placement 40 can be auditory cue in that the indicia 40 produces a single sound, a graduation of sound, or a plurality of different sounds. An example of auditory indicia of low placement 40D is an audible click resulting from the engagement of a locking mechanism, such as in FIG. 4. However, such auditory indicia 40 can form an auditory component of a sensor attached to the plunger 30, which completes a low ampere circuit which induces an audible alarm via sound generator attached to a power source positioned within the applicator.

Moreover, the indicia of low placement 40 can be a tactile cue, such as a plurality of holes, depressions of pits, channels, changing surface texture, coefficient of friction, on the insertion member 20 or a plurality of raised portions or a collar portion on plunger portion 30. Other tactile indicia of low placement 40 can be in the form of a plurality of holes in the plunger 30 or a plurality of raised portions in the plunger.

The insertion member 20 and the plunger 30 can be in the form of a spirally wound, convolutely wound or longitudinally seamed hollow tube, which is formed from paper, paperboard, cardboard or a combination thereof. The insertion member 20 and plunger 30 can also be injection molded, extruded or formed from flexible plastic, such as thermoformed from plastic sheet or folded or wound from plastic film. The insertion member 20 and plunger 30 can also be formed from a combination of paper and plastic.

The size of the insertion member 20, also commonly referred to as outer tube, can be determined primarily by the dimensions of the tampon 36. Specifically, the diameter of the insertion member 20 can be varied to accommodate different absorbency tampons 36. The inside diameter of the insertion member 20 is usually less than about 0.75 inches (about 19 mm) and less than about 0.625 inches (about 16 mm). Although the diameter of tampons 36 does vary, most tampons 36 utilized by women have an external diameter of less than about 0.75 inches (about 19 mm). However, if one desired to use this invention to administer medication to an animal, such as a farm animal or other mammal, larger size tampons 36, which would require insertion members with a larger diameter, could be used. Moreover, the inner diameter of the insertion member 20 can have varying diameters and shapes to conform to the profiled shape of the enclosed tampon 36. Generally, the inner diameter of the insertion member 20 should be suitably greater than the diameter of the tampon 36 to prevent the outer member 20 from interfering with the expulsion of the tampon 36 from the insertion member 20. The plunger 30 has a smaller diameter relative to the insertion member 20. The plunger 30 is dimensioned to slidably move within the insertion member 20, with minimal clearance therebetween.

The insertion member 20 and the plunger 30 have walls with a predetermined thickness of about 0.1 mm to about 2 mm. The walls of the insertion member 20 and the plunger 30 can be constructed from a single ply of material or be formed from two or more plies that are bonded together to form a laminate.

The use of two or more plies or layers is typical for making the insertion member 20 and the plunger 30 because it enables the manufacturer to use certain materials in the various layers that can enhance the performance of the tampon applicator 10. When two or more plies are utilized, all the plies can be spirally wound, convolutely wound or longitudinally seamed to form an elongated cylinder. For example, in some embodiments the wall can be constructed using a smooth thin ply of material on the outside or exterior surface that surrounds a coarser and possibly thicker ply. In the embodiments that contain at least three plies, the middle ply can be the thicker ply and the interior and exterior plies can be smooth and/or slippery to facilitate expulsion of the tampon 36 and to facilitate insertion of the insertion member 20 into a woman's vagina, respectively. By sandwiching a thick, coarser ply of material between two thin, smooth plies, an inexpensive insertion member 20 can be provided which is very functional. The wall can contain one to four plies, although more plies can be utilized if desired. As well, the ends of the insertion member 20 can be lipped.

An adhesive, such as glue, or heat, pressure, ultrasonics, etc, can hold the plies forming the wall together. The adhesive can be either water-soluble or water-insoluble. A water-soluble adhesive is for environmental reasons in that the wall will quickly break apart when it is immersed in water. Such immersion will occur should the insertion member 20 and plunger 30 be disposed of by flushing it down a toilet. Exposure of the insertion member 20 and plunger 30 to a municipal's waste treatment plant wherein soaking in water, interaction with chemicals and agitation all occur will cause the wall to break apart and evenly disperse in a relatively short period of time.

Alternatively, the material can be overlapped into a tubular configuration. Spirally or convolutely winding the insertion member 20 and/or plunger 30 into a cylindrical tube is especially advantageous when the insertion member 20 and/or plunger 30 are formed from a laminate.

The insertion member 20 is sized and configured to house an insertable element, such as an absorbent tampon 36. As stated above, the insertion member 20 should have a substantially smooth exterior surface or exterior surface that exerts low drag with vaginal body tissue that will facilitate insertion of the insertion member 20 into a woman's vagina. When the exterior surface is smooth and/or slippery, the insertion member 20 will easily slide into a woman's vagina without subjecting the internal tissues of the vagina to abrasion. The insertion member 20 can be coated to give it a high slip characteristic. Wax, polyethylene, a combination of wax and polyethylene, cellophane, clay, mica and other lubricants are representative coatings that can be applied to the insertion member 20 to facilitate comfortable insertion.

The applicator 10 of the present invention can have geometries or cross-sections that are useful to contain the object to be inserted. Often, the shape of the tampon 36 contained suggests the shape of the insertion member 20, but departures from this general rule can be made such that a cylindrical tampon 36 can be house in a rectangular shaped applicator, for example. The insertion member 20 and plunger 30 can take on numerous cross-sectional shapes including without limitations, circular, oval, polygonal (e.g. trapezoidal, rectangular, triangular) and the like. In addition the insertion member 20 and plunger 30 can be substantially elongated, such as in a linear fashion like TAMPAX PEARL SUPER, curved or flexible, or it can take on other shapes that are apparent to one of ordinary skill in the art. In addition the insertion member 20 and plunger 30 can be substantially elongated, curved or flexible, or it can take on other shapes that are apparent to one of ordinary skill in the art. Some examples of applicator shapes are described in WO 2004/024193 published by Lecan, et al. on Mar. 25, 2004 and European Patent Application No, 1101473 published by Mitsuhiro, et al on May 23, 2001.

The insertion end of the insertion member 30 can be open-ended or closed ended and can comprise petals, corrugations, pleats, or a film cap. During insertion, when the tampon 36 is pushed upward by the plunger 30, the petals open and to let the tampon 36 through, into the vagina.

The gripper end of insertion member 30 can comprise a fingergrip 46 configuration that is located on the insertion member, which allows the consumer to more securely hold the applicator during insertion of a material into the body cavity. Various fingergrip 46 configurations have been proposed to facilitate the handling of the applicator and to improve the insertion experience. One approach is a tampon applicator having an integral fingergrip 46 that is formed by embossing the outside surface of the insertion member of the tampon applicator. The embossed portion of the applicator can take the form of a series of circumferential rings or a series of discrete raised dots. Examples of such fingergrips 46 be found in U.S. Pat. No. 6,045,526 issued to Jackson; U.S. Pat. No. 5,395,308 issued to Fox, et al.; U.S. Pat. No. 5,290,501 issued to Klesius; U.S. Pat. No. 4,573,964 issued to Huffman; U.S. Pat. No. 4,573,963 issued to Sheldon; U.S. Pat. No. 4,891,042 issued to Nelvin, et al.; U.S. Pat. No. 4,412,833 issued to Weigner, et al.; U.S. Pat. No. 3,895,634 issued to Berger; U.S. Pat. No. 3,628,533 issued to Berger; U.S. Pat. No. 3,628,533 issued to Leyer; U.S. Pat. No. 2,922,423 issued to Rickard; U.S. Pat. No. 2,587,717 issued to Fourness; U.S. Pat. No. 2,489,502 issued to Ruth, U.S. Pat. No. 6,685,787 and U.S. Pat. No. 6,685,788 both issued to Linares, et al.

As well, the plunger 30 of the applicator 10 of the present invention can have a similar fingergrip portion or gripping means. The fingergrip on the plunger 30 can be any type of embossment or depression known in the art. An example of a fingergrip portion on a plunger 30 shown as a substantially flattened portion of the plunger as described in U.S. Pat. No. 3,068,867 issued to Bletzinger, et al on Dec. 18, 1962, a series of circumferential indentations to assist in gripping as described in the European Patent Application publication number 034922 published by Alexander, et al. on Mar. 1, 1990, or a pair diametrically disposed and axially extending guide channels on the plunger as shown in U.S. Pat. No. 3,196,873 issued to Bletzinger, et al on Aug. 27, 1962.

The plunger 30 of the applicator 10 of the present invention can be hollow or solid. An example of a solid plunger 30 is one that is rod shaped, as described in U.S. Pat. No. 3,068,867 issued to Bletzinger, et al on Dec. 18, 1962. The plunger 30 of the present invention can range from 45 to 100 mm in length measured from the first end 26 of the plunger 30 to the second end 28 of the plunger 30. The plunger 30 also comprises an indicia of low placement 40 that is located between the first end 26 and the second end 28.

The insertion member 20 and the plunger 30 can combine to have a locking mechanism 48 such as in tampon applicator 10 as disclosed in U.S. Pat. No. 6,019,744, with a locking mechanism comprising a tongue shaped retention element that is integrally connected to the plunger 30 so as to be bendable and project through the longitudinal slit of the insertion member 20. Another approach is a tampon applicator 10 as disclosed in U.S. Pat. No. 6,450,986, comprising a plunger 30 having a rim comprising a shoulder projecting radially outward from the plunger and a return margin. The rim creates an interference with the barrel of the insertion member 20 to provide a stop.

The applicator 10 of the present invention could be part of a kit for providing low placement of the tampon. The kit can be a package that comprises at least one applicator 10 capable of housing a tampon 36. The applicator 10 within the kit comprises an insertion member 20, a plunger 30 and tampon 36, positioned within the insertion member 20. The insertion member 20 comprises an insertion end 22 opposed to a gripper end 24. The plunger 30 has a longitudinal axis L, a first end 26, a second end 28 and an indicia of low placement 40. The indicia of low placement 40 is positioned between the first end 22 opposed to the second end 28 of the plunger 30 when the applicator 10 is in a pre-insertion position.

The kit also comprises a set of instructions in association with a package. The set of instructions comprises instructions for placing the tampon 36 in a low placement position within the vagina. The package comprises the following written instructions: First, the instructions state that that the consumer should grasp the insertion member 20 with her fingers at the gripper end 24. After that, the instructions state for the consumer to position the insertion member 20 at or proximate to the vaginal opening and slide the insertion member 20 into the vagina until her fingers are proximate with or touch her body. Next, the instructions state that the consumer to push the second end 28 of the plunger 30 in closer proximity towards the gripper end 24 of the insertion member 20 such that the indicia of low placement 40 on the plunger 30 is flush with the gripper 24 end of the insertion member 20.

The applicator of the present invention is made by first providing an insertion member 20 having an insertion end 22 and a gripper end 24. Next, a plunger 30 is provided having a first end 26, and a second end 28. Next, the plunger is inserted into the insertion member 20. In some embodiments, the locking mechanism 48 is arranged for the plunger 30 to be slideably introduced into the insertion member 20. The indicia of low placement 40 can be produced during or after the formation of the plunger 30. In the case where the indicia 40 is a line, color or a change or graduation in color and pattern, the plunger 30 can be sprayed, soaked, painted, dyed, screen printed, laser printed, or otherwise marked. Any dye, paint or pigment can be used. Suitable dyes, paint and pigments are permanent, so as to not to stain the user or garments of wearer, and nontoxic and nonirritating to the wearer.

The indicia for low placement 40 is positioned on the plunger 30 to insure that the tampon 36 is inserted at a low placement position in the vagina. A low placement position refers to a position of the tampon 36 inside the vaginal cavity, wherein the insertion end 54 of the tampon 36 is positioned generally below the cervix and the withdrawal end 56 of the tampon 36 is positioned generally above the hymenal ring. To achieve a low placement position of the tampon 36, the indicia for low placement 40 must be in the correct position on the plunger 30. If the indicia 40 is put in an incorrect position, following the plunging of the applicator 10, the tampon 36 can be placed too high in the vagina or too low in the vagina. Thus, for the purposes of the present invention, the indicia for low placement 40 is positioned on the plunger 30 at a distance Ip. The measured distance Ip at which the indicia for low placement 40 are placed can be derived by either one of the following equations:
Ip=A+TL−(M−X) and (1)
Ip=EP+TL−M (2)

In this equation, the value of “A” describes the distance that the tampon 36 must move for the tampon 36 to be in a low placement position. It follows that the value of “A” in the equation, is the distance between the insertion end 54 of the tampon 36 at a pre-insertion position, which is at the labia majora, and the withdrawal end 56 of the tampon 36 within the vagina after insertion. The value of “A” can vary depending on the target depth of insertion for the tampon 36 to be in low placement position within the vagina.

TABLE 1 shows the values of A. The values for A in TABLE 1, below, are based on the data collected by the applicants from the dozens of panelists according to MRI and testing. The value of A₁ describes the distance that the tampon 36 must move if the target depth is at or just above the hymenal ring. A₁ is the distance between the insertion end 54 of the tampon 36 at a pre-insertion position at the labia majora and the withdrawal end 56 of the tampon 36 at the hymenal ring. The value of A₂ describes the distance that the tampon 36 must move if the target depth is 5 mm above the hymenal ring. A₂ is the distance between the insertion end 54 of the tampon 36 at a pre-insertion position at the labia majora and the withdrawal end 56 of the tampon 36 at a distance is 5 mm above the hymenal ring. The value of A₃ describes the distance that the tampon 36 must move if the target depth is 10 mm above the hymenal ring. A₃ is the distance between the insertion end 54 of the tampon 36 at a pre-insertion position at the labia majora and the withdrawal end 56 of the tampon 36 at a distance of 10 mm above the hymenal ring. The value of A₄ describes the distance that the tampon 36 must move if the target depth is 15 mm above the hymenal ring. A₄ is the distance between the insertion end 54 of the tampon 36 at a pre-insertion position at the labia majora and the withdrawal end 56 of the tampon 36 at a distance of 15 mm above the hymenal ring. The value of A is greater than about 21 mm and less than about 36 mm.

TABLE 1
Values of A

| | |
|---|---|
| A₁ | 21 mm |
| A₂ | 26 mm |
| A₃ | 31 mm |
| A₄ | 36 mm |

In this equation, “TL” describes the length in mm of the tampon 36 measured from the insertion end 54 of the tampon 36 to the withdrawal end 56 of the tampon 36.

In this equation M−X describes the length of the insertion member 20 as it is placed beyond the labia majora during the insertion of the applicator 10 in the vagina. M is the length of the insertion member 20 from insertion end 22 to the gripper end 24 of the insertion member 20. Note in an open ended applicator, as in FIG. 5, M is measured from the insertion end 54 of the tampon 36 to the gripper end 34 of the insertion member 20. X is the length of the fingergrip from insertion end 58 of the fingergrip 46 to the gripper end 24 of the insertion member 20. Typically, X is 17 mm, the assumed length a typical consumer uses to grip the insertion member 20 at the gripper end 24.

EP or the “effective plunge” is the value of A+X. EP can be substituted for A+X in, the equation (1) Ip=A+TL−M+X, to derive the equation (2) Ip=EP+TL−M. In the value of EP, X is 17 mm, the assumed length a typical consumer uses to grip the insertion 20 at the gripper end 24. If the actual fingergrip 46 on the applicator 10 as measured from insertion end 58 to the gripper end 24 of the insertion member 20 is not equal to 17 mm the EP can be modified. “EP modified” or “EPₘ” can be calculated by the equation (A−X)+17 mm; where X is the measured length of the fingergrip from insertion end 58 of the fingergrip 46 to the gripper end 24 of the insertion member 20. Typically, the EP ranges from 38-60 mm or from 43-60 mm.

Values of Ep (x = 17 mm)

| | |
|---|---|
| A₁ | 38 mm |
| A₂ | 43 mm |
| A₃ | 48 mm |
| A₄ | 53 mm |

The placement of the indicia of low placement 40 is dependent on both the tampon length and the effective plunge length. The term “combined effective plunge” or “CEP” as used herein is the mathematical total of TL added to the EP. The CEP in the present application is less than 103 mm. Alternatively, the CEP is less than 99 mm.

The value of “Ip” that results from either equation describes the length of the plunger 30 that should be used to achieve a low placement position of the tampon 36 after proper insertion in the vagina. Thus, Ip is the measured distance at which the indicia for low placement 40 is placed on the plunger 30, as measured from the gripper end 24 of the insertion member 20 toward the second end 28 of the plunger 30. To measure Ip properly, so that the indicia for low placement 40 is in the correct position, the applicator must be in a pre-insertion position. The term “pre-insertion position,” as used herein refers to the arrangement of the applicator 10 prior to insertion, where the first end 26 of the plunger 30 is contact with the withdrawal end 56 of the tampon 36 and that the insertion end 54 of the tampon 36 be proximate to the insertion end 22 of the insertion member 20, such that any additional movement of the plunger 30 would cause an equal and mutually corresponding movement of the tampon 36. Note that the applicator, as received by the consumer, may or may not be in the pre-insertion position. Thus, prior to measuring the Ip the applicator 10 must be placed in a pre-insertion position. FIG. 5 is a side view of the applicator 10 that has the insertion member 20 in cross-section, so one can view the position of the tampon 36 and plunger 30 within the insertion member 20 when the applicator 10 is in the pre-insertion position. The value of Ip is greater than about 21 mm+TL−(M−X) and less than about 36 mm+TL−(M−X).

FIG. 5 illustrates the values of TL, M, X, and Ip on an applicator 10 of the present invention.

When the indicia 40 is marked in the correct position, a low placement position of the tampon 36 will be achieved following proper plunging of the applicator by the consumer. During the plunge, the indicia of low placement 40 is a cue for the consumer to stop moving or sliding the second end 28 of plunger 30 towards the gripper end 24 of the insertion member 30, so that tampon 36 is placed in the desired position within the vagina.

### EXAMPLE 1

The applicator 10 of the present invention is comprised of plastic. The applicator 10 includes an insertion member 20 that has a length of the insertion member 20 from insertion end 22 to the gripper end 24 or M of 59 mm and a tampon 36 that has a length from insertion end 54 to withdrawal end 56 or TL, which is 42 mm when the applicator 10 is in the pre-insertion position. X is the length of the fingergrip from insertion end 58 of the fingergrip 46 to the gripper end 24 of the insertion member 20 and is 17 mm. Using the equation Ip=A+TL−M+X; the value of Ip would the following at various values of A that fall in the low placement range.

| The value of A | The value of Ip |
|---|---|
| A₁ = 21 mm | 21 mm |
| A₂ = 26 mm | 26 mm |
| A₃ = 31 mm | 31 mm |
| A₄ = 36 mm | 36 mm |

Note, that the Ip could be at or between any of the distances in the table for the tampon 36 to be inserted in a low placement position.

However, if one would want to place the withdrawal end 56 of the tampon 36 to be at the hymenal ring, the Ip would be 21 mm, so the starting point 50 of indicia of low placement 40 would be positioned on the plunger 30 at a distance of 21 mm as measured from the from the gripper end 24 of the insertion member 20 towards the second end 28 of the plunger 30. The indicia of low placement 40 is an audible click resulting from the engagement of a locking mechanism 48 that when engaged would be positioned on the plunger 30 at a distance of 21 mm as measured from the from the gripper end 24 of the insertion member 20 towards the second end 28 of the plunger 30.

### EXAMPLE 2

The applicator 10 of the present invention is comprised of plastic. The applicator 10 includes an insertion member 20 a length of the insertion member 20 from insertion end 22 to the gripper end 24 or M of 70 mm and a tampon 36 that has a length from insertion end 54 to withdrawal end 56 or TL, which is 48 mm when the applicator 10 is in the pre-insertion position. Using the equation Ip=EP+TL−M; the value of Ip would the following at various values of EP that fall in the low placement range.

| The value of EP | The value of Ip |
|---|---|
| 38 mm | 16 mm |
| 43 mm | 21 mm |
| 48 mm | 26 mm |
| 53 mm | 31 mm |

Note, that the Ip could be at or between any of the distances in the table for the tampon 36 to be inserted in a low placement position.

However, if one would want the withdrawal end 56 of the tampon 36 to be at a distance 10 mm above the hymenal ring, the EP is 48 mm and the Ip would be would be 26 mm. The indicia of low placement 40 can be a transition point between two textures on the surface of the plunger 30, such that 26 mm on the plunger 30, as measured from the gripper end 24 of the insertion member 20 is smooth and the remainder of the plunger, as measured from the gripper end 24 of the insertion member 20 to the second end 28 of the plunger 30 is a pebbled texture.

### EXAMPLE 3

The applicator 10 of the present invention is comprised of paperboard. The applicator 10 can include an insertion member 20 a length of the insertion member 20 from insertion end 22 to the gripper end 24 or M of 70 mm and a tampon 36 that has a length from insertion end 54 to withdrawal end 56 or TL, which is 48 mm when the applicator 10 is in the pre-insertion position. X is the length of the fingergrip from insertion end 58 of the fingergrip 46 to the gripper end 24 of the insertion member 20 and is 17 mm. Using the equation Ip=A+TL−M+X; the value of Ip would the following at various values of A that fall in the low placement range.

| The value of A | The value of Ip |
|---|---|
| A₁ = 21 mm | 16 mm |
| A₂ = 26 mm | 21 mm |
| A₃ = 31 mm | 26 mm |
| A₄ = 36 mm | 31 mm |

Note, that the Ip could be at or between any of the distances in the table for the tampon 36 to be inserted in a low placement position.

However, one could put two indicia for low placement 40 on the same applicator so the consumer could have a choice of insertion depths. The first indicia 40 would be for placing the withdrawal end 56 of the tampon 36 to be at a distance 5 mm above the hymenal ring, so the Ip is 21 mm. The indicia of low placement 40 is a 2 mm band of blue color encircling the perimeter of the plunger 30, the starting point 50 of the indicia 40 is positioned at a distance of 21 mm, as measured from the gripper end 24 of the insertion member 20 toward the second end of the plunger 30. The second indicia 40 would be for placing the withdrawal end 56 of the tampon 36 to be at a distance 10 mm above the hymenal ring, so the Ip is 26 mm. The indicia of low placement 40 is a 3 mm band of lavender color encircling the perimeter of the plunger 30, the starting point 50 of the indicia 40 starting point 50 is positioned at a distance of 26 mm, as measured from the gripper end 24 of the insertion member 20 toward the second end of the plunger 30.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An applicator capable of housing a tampon comprising:
an insertion member having a hollow interior, an insertion end opposed to a gripper end; a plunger slideably configured within said insertion member; said plunger having a longitudinal axis, a first end opposed to a second end and an indicia of low placement; said indicia of low placement being positioned between said first end and said second end of said plunger when said application is in a pre-insertion position.

2. The applicator of claim 1 wherein said indicia of low placement is positioned between said gripper end of said insertion member and said second end of said plunger when said applicator is in a pre-insertion position.

3. The applicator of claim 1 wherein said indicia of low placement is positioned at a distance Ip on said plunger as measured from said gripper end of said insertion member when said applicator is in a pre-insertion position according to a formula Ip=A+TL−(M−X).

4. The applicator of claim 1 further comprises an effective plunge (EP) of from about 38 mm to about 60 mm.

5. The applicator of claim 5 further comprising a tampon length (TL) ranges from about 30 mm to about 56.

6. The applicator of claim 1 further comprises a combined effective plunge (CEP) of less than 99

7. The applicator of claim 8 further comprising a tampon length (TL) of from about 30 mm to about 56.

8. The applicator of claim 1 further comprising a combined effective plunge (CEP) of less than 103.

9. The applicator of claim 9 further comprising a tampon length (TL) ranges from about 30 mm to about 56.

10. The applicator of claim 1 further comprises an effective plunge (EP) of about 38 mm to about 60 mm and a combined effective plunge (CEP) length of less than 103.

11. The applicator of claim 1 further comprises an effective plunge (EP) of about 43 mm to about 60 mm and a combined effective plunge (CEP) length of less than 99.

12. The applicator of claim 1 wherein said indicia of low placement comprises visual indicia.

13. The applicator of claim 1 wherein said indicia of low placement comprises tactile indicia.

14. The applicator of claim 1 wherein said indicia of low placement comprises auditory indicia.

15. The applicator of claim 1 wherein said applicator is comprised of plastic.

16. The applicator of claim 1 wherein said applicator is comprised of plastic.

17. A kit for low placement of tampon comprising:
(a) a package comprising at least one applicator capable of housing a tampon; said applicator comprising an insertion member having a hollow interior, an insertion end opposed to a gripper end; said plunger having a longitudinal axis, a first end opposed to a second end and an indicia of low placement; said indicia of low placement being positioned between said first end and said second end of said plunger when said application is in a pre-insertion position; and
(b) a set of instructions in association with said package; said set of instructions comprising an instruction for placing the tampon in a low placement position within the vagina.

18. A kit according to claim 8 wherein said of instruction comprising the steps of:
a. grasping said insertion member at said gripper end;
b. position the insertion member at the vaginal opening and slide the insertion member into the vagina until your fingers touch the body;
c. pushing said second end of said plunger in closer proximity towards said gripper end of said insertion member such that said indicia of low placement on said plunger is flush with said gripper end of said insertion member.
